(19) United States
(12) Patent Application Publication
(10) Pub. No.: US 2025/0057403 A1
(43) Pub. Date: Feb. 20, 2025

(54) ENDOSCOPE

(71) Applicant: FUJIFILM Corporation, Tokyo (JP)

(72) Inventors: Takuro ASAOKA, Ashigarakami-gun, Kanagawa (JP); Makoto SUGIZAKI, Ashigarakami-gun, Kanagawa (JP)

(73) Assignee: FUJIFILM Corporation, Tokyo (JP)

(21) Appl. No.: 18/801,995

(22) Filed: Aug. 13, 2024

(30) Foreign Application Priority Data
Aug. 18, 2023 (JP) 2023-133414

(51) Int. Cl. A61B 1/04; A61B 1/00

(52) U.S. Cl. CPC A61B 1/042; A61B 1/00114

(57) ABSTRACT

An endoscope includes an insertion part including an imaging optical system, a tubular support member, an imaging element, a first circuit substrate, a second circuit substrate, a cable, a covering member, and a distal end member as defined herein, the support member includes a circular tubular portion having a circular tubular shape that supports the imaging optical system and a rectangular tubular portion having a rectangular tubular shape that supports the imaging element, the circular tubular portion is supported by the hole portion of the distal end member, a clearance is provided between the distal end member and the covering member or the rectangular tubular portion of the support member, the covering member covers at least a part of the rectangular tubular portion, and a diameter of a circumscribed circle of the rectangular tubular portion is equal to or larger than an outer diameter of the circular tubular portion.

### CROSS-REFERENCE TO RELATED APPLICATIONS

The present application claims priority under 35 U.S.C. § 119 to Japanese Patent Application No. 2023-133414, filed on Aug. 18, 2023. The above application is hereby expressly incorporated by reference, in its entirety, into the present application.

### BACKGROUND OF THE INVENTION

### 1. Field of the Invention

The present invention relates to an endoscope.

### 2. Description of the Related Art

JP6038424B discloses an imaging module comprising a chip size package in which a plurality of connection lands are disposed on a back surface side of a light receiving section of an imaging element, a circuit substrate to which a plurality of connection electrodes are electrically and mechanically connected via the connection lands of the chip size package and bumps, and an underfill agent filled in a gap between the chip size package and the circuit substrate.

JP6697244B discloses a circuit substrate unit in which a first signal cable is connected to a land group on a front row side on a surface of the circuit substrate configured to mount an imaging element on the surface thereof, and the first signal cable that is connected to the land group on the front row side and that extends rearward in an axial direction and a second signal cable that is connected to a land group on a rear row side and that is arranged in tandem with the first signal cable in the axial direction are disposed such that the first signal cable and the second signal cable overlap each other in a direction perpendicular to the surface of the circuit substrate.

JP2005-95432A discloses an imaging apparatus comprising a solid-state imaging element, an objective lens unit for forming a subject image on the solid-state imaging element, and a holder that holds the solid-state imaging element and the objective lens unit, in which a region other than a light receiving section region of the solid-state imaging element, which is in a substantially parallel relationship with the light receiving section region of the solid-state imaging element, is brought into contact with the holder, and the light receiving section region and an optical axis center of the objective lens unit are disposed in a substantially perpendicular relationship.

### SUMMARY OF THE INVENTION

The technology of the present disclosure provides an endoscope that can effectively utilize a space inside an insertion part.

An endoscope according to one aspect of the technology of the present disclosure comprises an insertion part that is to be inserted into a subject, in which the insertion part includes an imaging optical system, a tubular support member that supports the imaging optical system within the tubular support member, an imaging element that is provided on a proximal end side of the insertion part with respect to the imaging optical system and that is supported by the support member, a first circuit substrate that has a first substrate surface facing a surface of the imaging element on the proximal end side of the insertion part and a second substrate surface on a side opposite to the first substrate surface and that is connected to the imaging element at the first substrate surface, a second circuit substrate that is provided on the proximal end side of the insertion part with respect to the first circuit substrate, that has a third substrate surface intersecting with the first substrate surface and a fourth substrate surface on a side opposite to the third substrate surface, and that is connected to the second substrate surface at at least one of the third substrate surface or the fourth substrate surface, a cable that is connected to at least one of the third substrate surface or the fourth substrate surface of the second circuit substrate, a covering member that covers a part of the support member, the imaging element, the first circuit substrate, the second circuit substrate, and the cable, and a distal end member having a hole portion into which the support member and the imaging optical system are inserted, the support member includes a circular tubular portion having a circular tubular shape that supports the imaging optical system and a rectangular tubular portion having a rectangular tubular shape that supports the imaging element, the circular tubular portion is supported by the hole portion of the distal end member, a clearance is provided between the distal end member and the covering member or the rectangular tubular portion of the support member, the covering member covers at least a part of the rectangular tubular portion, and a diameter of a circumscribed circle of the rectangular tubular portion is equal to or larger than an outer diameter of the circular tubular portion.

An endoscope according to another aspect of the technology of the present disclosure comprises an insertion part that is to be inserted into a subject, in which the insertion part includes an imaging optical system, a tubular support member that supports the imaging optical system within the tubular support member, an imaging element that is provided on a proximal end side of the insertion part with respect to the imaging optical system and that is supported by the support member, a first circuit substrate that has a first substrate surface facing a surface of the imaging element on the proximal end side of the insertion part and a second substrate surface on a side opposite to the first substrate surface and that is connected to the imaging element at the first substrate surface, a cable that is provided on the proximal end side of the insertion part with respect to the first circuit substrate and that is electrically connected to the first circuit substrate, a covering member that covers a part of the support member, the imaging element, the first circuit substrate, and the cable, and a distal end member having a hole portion into which the support member and the imaging optical system are inserted, the support member includes a circular tubular portion having a circular tubular shape that supports the imaging optical system and a rectangular tubular portion having a rectangular tubular shape that supports the imaging element, the circular tubular portion is supported by the hole portion of the distal end member, a clearance is provided between the distal end member and the covering member or the rectangular tubular portion of the support member, a plurality of terminals electrically connected to the imaging element are provided on the first substrate surface, and in a case of being viewed in an axial direction of the insertion part, a total area of the plurality of terminals is 30% or less of an area of an end surface of the imaging element on a distal end side of the insertion part.

According to the technology of the present disclosure, the space inside the insertion part of the endoscope can be effectively utilized.

### BRIEF DESCRIPTION OF THE DRAWINGS

FIG. 1 is a schematic view showing an endoscope 1 according to an embodiment of the technology of the present disclosure.

FIG. 2 is a schematic cross-sectional view showing an internal configuration of a distal end portion 7 of the endoscope 1 shown in FIG. 1.

FIG. 3 is a schematic cross-sectional view taken along line A-A of FIG. 2.

FIG. 4 is a schematic cross-sectional view taken along line B-B of FIG. 2.

FIG. 5 is a schematic view of an imaging element 72 as viewed from a distal end side in an axial direction.

FIG. 6 is a schematic view of the imaging element 72 and a first circuit substrate 73 as viewed from the distal end side in the axial direction.

FIG. 7 is a schematic view showing a modification example of a B-B cross section of FIG. 2.

FIG. 8 is a schematic cross-sectional view taken along line C-C of FIG. 2.

FIG. 9 is a schematic view showing a modification example of a covering member 76 and is a view corresponding to FIG. 4.

FIG. 10 is a schematic cross-sectional view showing a first modification example of the distal end portion 7.

FIG. 11 is a schematic cross-sectional view showing a second modification example of the distal end portion 7.

FIG. 12 is a schematic cross-sectional view showing a third modification example of the distal end portion 7.

FIG. 13 is a schematic cross-sectional view showing a fourth modification example of the distal end portion 7.

### DESCRIPTION OF THE PREFERRED EMBODIMENTS

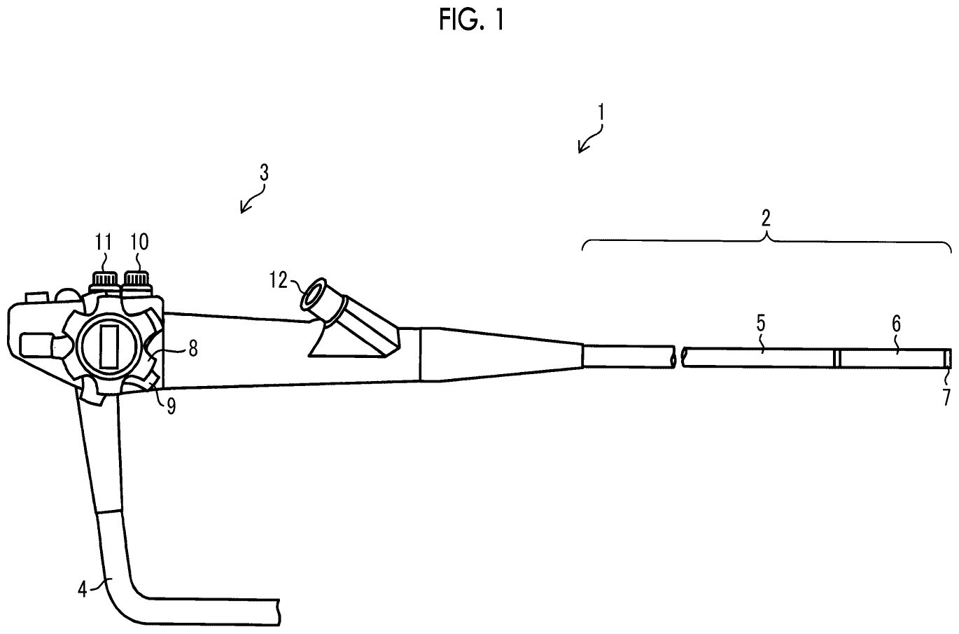

FIG. 1 is a schematic view showing an endoscope 1 according to an embodiment of the technology of the present disclosure. The endoscope 1 in FIG. 1 comprises an elongated insertion part 2 that is inserted into a subject, an operating part 3 that is connected to a proximal end of the insertion part 2 and that is used for gripping and operating the endoscope 1, and a universal cord 4 that connects the endoscope 1 to a system constituent device such as a light source device and a processor device (not shown). The endoscope 1 is not particularly limited, and is, for example, an upper endoscope for observing a stomach or a duodenum, a lower endoscope (colonoscope) for observing a large intestine, a bronchoscope, or the like.

The insertion part 2 is composed of a soft portion 5, a bendable portion 6, and a distal end portion 7 that are consecutively provided in order from the proximal end toward a distal end. The soft portion 5 is flexible and is bendable in any direction along an insertion path of the insertion part 2. The operating part 3 is provided with angle knobs 8 and 9, a treatment tool inlet port 12, an air/water supply button 10, a suction button 11, and the like.

The bendable portion 6 is bent in each of up-down and left-right directions by the operation of each of the angle knobs 8 and 9. A treatment tool, such as a forceps, is inserted from the treatment tool inlet port 12 and is led out from a forceps port (not shown) provided in the distal end portion 7. In addition, the distal end portion 7 is provided with an observation window (not shown) that images an observation target site in a body and an illumination window (not shown) that irradiates the observation target site with illumination light.

The insertion part 2 is inserted into a subject by moving along an axial direction thereof, and the angle knobs 8 and 9 of the operating part 3 are rotationally operated to bend the bendable portion 6 of the insertion part 2 in the up-down and left-right directions. Accordingly, the distal end portion 7 of the insertion part 2 can be directed to a desired direction in the body, and an observation image can be acquired by using the observation window provided in the distal end portion 7. Hereinafter, the axial direction of the insertion part 2 will be simply referred to as an axial direction. In addition, the operating part 3 side in the axial direction will be referred to as a proximal end side, and the distal end portion 7 side in the axial direction will be referred to as a distal end side.

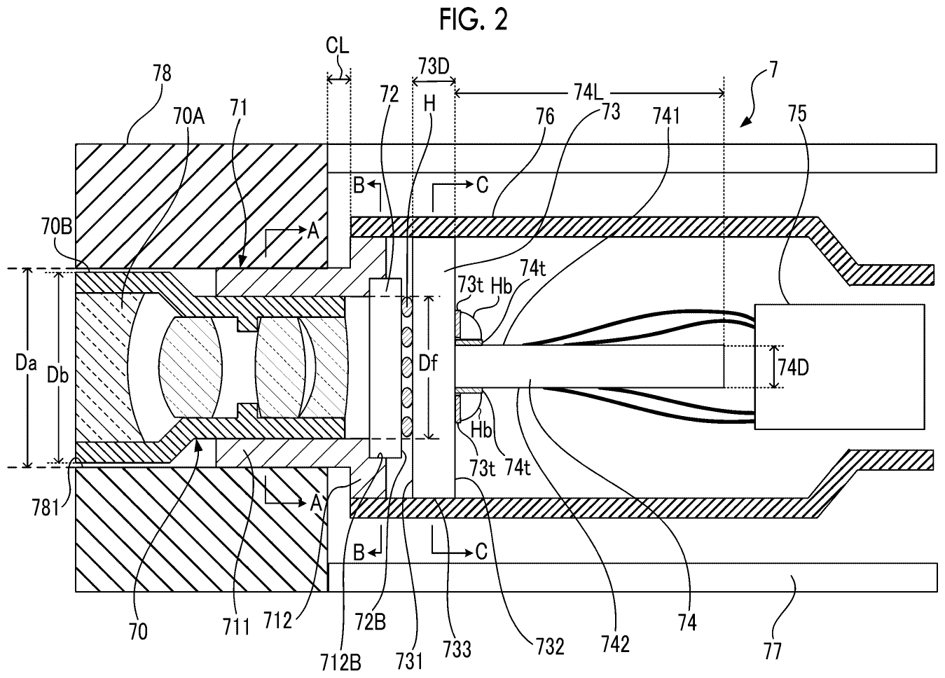

FIG. 2 is a schematic cross-sectional view showing an internal configuration of the distal end portion 7 of the endoscope 1 shown in FIG. 1.

As shown in FIG. 2, the distal end portion 7 includes a distal end member 78 provided on the most distal end side, and an exterior member 77 having a circular tubular shape provided on the proximal end side with respect to the distal end member 78. The distal end member 78 and the exterior member 77 have a cylindrical outer shape, and constituent elements required for imaging, a light guide, a treatment tool channel, and the like are provided inside the distal end member 78 and the exterior member 77. The distal end member 78 is provided with a cylindrical hole portion 781 that extends from an end surface on the proximal end side to an end surface on the distal end side.

The distal end portion 7 comprises, as the constituent elements required for imaging, an imaging optical system 70, a tubular support member 71 that supports the imaging optical system 70 within the tubular support member 71, an imaging element 72 that is provided on the proximal end side with respect to the imaging optical system 70 and that is supported by the support member 71, a first circuit substrate 73, a second circuit substrate 74, a cable 75, and a covering member 76 that covers a part of the support member 71, the imaging element 72, the first circuit substrate 73, the second circuit substrate 74, and the cable 75.

In the shown example, the imaging optical system 70 includes a lens frame 70B having a circular tubular shape of which a distal end side has a larger diameter than a proximal end side, and a lens group (in the shown example, four lenses 70A) supported inside the lens frame 70B. FIG. 2 shows a maximum diameter Db of the imaging optical system 70. In the present specification, the maximum diameter Db is defined as an outer diameter of the imaging optical system 70.

The support member 71 includes a circular tubular portion 711 having a circular tubular shape that supports a small-diameter portion of the lens frame 70B on the proximal end side inside the support member 71, and a rectangular tubular portion 712 having a rectangular tubular shape that supports the imaging element 72. The circular tubular portion 711 is disposed on the distal end side with respect to the rectangular tubular portion 712.

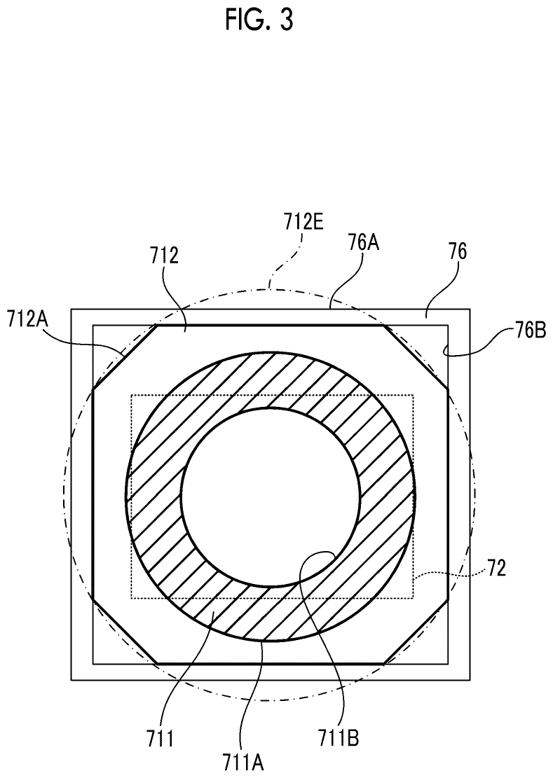

FIG. 3 is a schematic cross-sectional view taken along line A-A of FIG. 2. In FIG. 3, the imaging optical system 70 and the distal end member 78 are not shown.

As shown in FIG. 3, the circular tubular portion 711 includes an outer peripheral surface 711A having a circular cross section perpendicular to the axial direction and an inner peripheral surface 711B having a circular cross section perpendicular to the axial direction. In the inner peripheral surface 711B, the small-diameter portion on the proximal end side of the lens frame 70B constituting the imaging optical system 70 is supported. FIG. 2 shows an outer diameter Df of a portion (the small-diameter portion of the lens frame 70B) of the imaging optical system 70 which is supported by the inner peripheral surface 711B.

As shown in FIG. 2, a portion of the imaging optical system 70 located outside the support member 71 and the circular tubular portion 711 of the support member 71 are inserted into the hole portion 781 of the distal end member 78, and the circular tubular portion 711 is supported by an inner peripheral surface of the hole portion 781. An outer diameter Da of the circular tubular portion 711 (a diameter of the outer peripheral surface 711A in FIG. 3, see FIG. 2) is larger than the maximum diameter Db of the imaging optical system 70. As a result, the imaging optical system 70 is configured such that the imaging optical system 70 is not directly supported by the hole portion 781 of the distal end member 78.

A support form between the circular tubular portion 711 and the hole portion 781 and a support form between the imaging optical system 70 and the circular tubular portion 711 are not particularly limited, and, for example, a support form such as any one of fitting, adhesion, or screwing or a combination of two or more of these can be adopted.

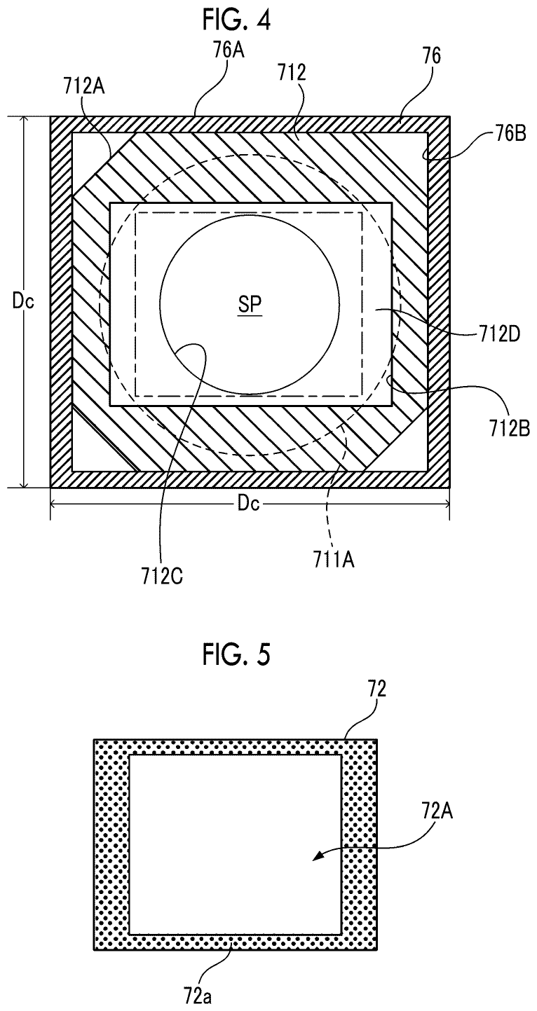

FIG. 4 is a schematic cross-sectional view taken along line B-B of FIG. 2. In FIG. 4, the imaging optical system 70 and the distal end member 78 are not shown. As shown in FIGS. 3 and 4, an outer peripheral surface 712A of the rectangular tubular portion 712 forms a polygon with four or more sides (in the examples of FIGS. 3 and 4, an octagon in which four corner portions of a square are chamfered) in a cross section perpendicular to the axial direction. It should be noted that the outer peripheral surface 712A may have a shape in which corner portions of a polygon with four or more sides are chamfered in a curved shape (R-shape) instead of a straight line shape.

As shown in FIG. 4, a rectangular plate-shaped recessed portion 712B is provided on an end surface of the rectangular tubular portion 712 on the proximal end side. An opening 712C communicating with an inner space SP of the circular tubular portion 711 is formed on a bottom surface of the recessed portion 712B.

FIG. 5 is a schematic view of the imaging element 72 as viewed from the distal end side in the axial direction. The imaging element 72 is a packaged charge-coupled device (CCD) image sensor, a complementary metal-oxide-semiconductor (CMOS) image sensor, or the like. As shown in FIG. 5, the imaging element 72 has a rectangular shape as viewed in the axial direction. The imaging element 72 is supported by the rectangular tubular portion 712 in the recessed portion 712B shown in FIG. 4.

As shown in FIG. 4, an adhesive region 712D (a region between a rectangular frame indicated by a one-dot chain line in the drawing and an outer peripheral edge of the recessed portion 712B) to which the imaging element 72 is adhered is provided on the bottom surface of the recessed portion 712B. The imaging element 72 is supported by the rectangular tubular portion 712 by being adhered to the recessed portion 712B in the adhesive region 712D. The imaging element 72 may be supported by the rectangular tubular portion 712 by being fitted into the recessed portion 712B. The imaging element 72 may be supported by the rectangular tubular portion 712 by being fitted into the recessed portion 712B and further being adhered to the adhesive region 712D. The imaging element 72 may be supported by the rectangular tubular portion 712 by adhering a side surface thereof to a side wall of the recessed portion 712B.

A rectangular frame-shaped peripheral region 72a (see FIG. 5) of the end surface 72A on the distal end side of the imaging element 72 is adhered to the adhesive region 712D shown in FIG. 4. From the viewpoint of realizing a reduction in diameter of the insertion part 2 and increasing a light receiving area of the imaging element 72, it is preferable that an area of the peripheral region 72a is 50% or less of an area of the end surface 72A.

As shown in FIG. 2, the first circuit substrate 73 has a first substrate surface 731 facing the end surface 72B of the imaging element 72 on the proximal end side, and a second substrate surface 732 on a side (proximal end side) opposite to the first substrate surface 731, and is connected to the imaging element 72 at the first substrate surface 731. The first circuit substrate 73 is a rigid substrate having a bending rigidity larger than that of the imaging element 72, and, for example, has a plate shape in which a plurality of wiring lines are laminated through an insulating layer from the first substrate surface 731 toward the second substrate surface 732. As the first circuit substrate 73, for example, a ceramic substrate, a glass substrate, a silicon substrate, or the like is used. The first circuit substrate 73 is disposed in a state in which the first substrate surface 731 and the second substrate surface 732 intersect with the axial direction (in the example of FIG. 2, a state in which the first substrate surface 731 and the second substrate surface 732 are perpendicular to the axial direction).

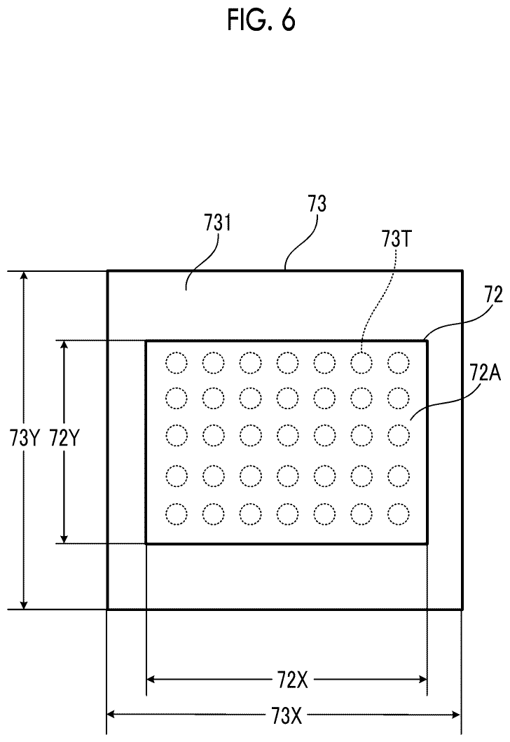

A plurality of terminals are disposed in a two-dimensional manner on the end surface 72B of the imaging element 72. In addition, terminals 73T (see FIG. 6) corresponding to the plurality of terminals of the imaging element 72 are disposed on the first substrate surface 731 of the first circuit substrate 73 in a two-dimensional manner. Each terminal of the end surface 72B of the imaging element 72 and the terminal 73T, which corresponds to each terminal, on the first substrate surface 731 of the first circuit substrate 73 are electrically and mechanically connected to each other by a solder ball H. It is preferable that an underfill agent is provided between the imaging element 72 and the first circuit substrate 73 in order to make a connection between the imaging element 72 and the first circuit substrate 73 strong.

FIG. 6 is a schematic view of the imaging element 72 and the first circuit substrate 73 as viewed from the distal end side in the axial direction. A plurality of (35 in the shown example) terminals 73T are disposed in a two-dimensional manner on the first substrate surface 731 of the first circuit substrate 73. A total area of the plurality of terminals 73T as viewed in the axial direction is, for example, 30% or less of an area of the end surface 72A of the imaging element 72 on the distal end side. In this way, it is possible to prevent a circuit formed on the first circuit substrate 73 from being complicated. In a case where the total area of the plurality of terminals 73T exceeds 30% of the area of the end surface 72A of the imaging element 72, a mechanical connection between the imaging element 72 and the first circuit substrate 73 can be made stronger.

FIG. 6 shows a length 72X of a long side of the imaging element 72, a length 73X of two sides along the long side of the first circuit substrate 73 among four sides of the first circuit substrate 73, a length 72Y of a short side of the imaging element 72, and a length 73Y of two sides along the short side of the first circuit substrate 73 among the four sides of the first circuit substrate 73.

As viewed in the axial direction, a size of the first circuit substrate 73 is larger than a size of the imaging element 72. The configuration in which the size of the first circuit substrate 73 is larger than the size of the imaging element 72 refers to any of a configuration in which the length 73X is larger than the length 72X and the length 73Y is larger than the length 72Y, a configuration in which the length 73X is the same as the length 72X and the length 73Y is larger than the length 72Y, or a configuration in which the length 73X is larger than the length 72X and the length 73Y is the same as the length 72Y.

In the example of FIG. 6, the length 73X is larger than the length 72X and the length 73Y is larger than the length 72Y, and as viewed in the axial direction, the entire imaging element 72 overlaps the first circuit substrate 73.

In addition, as viewed in the axial direction, the size of the first circuit substrate 73 is larger than the outer diameter Df shown in FIG. 2. The size of the first circuit substrate 73 referred to here means the length 73X and the length 73Y.

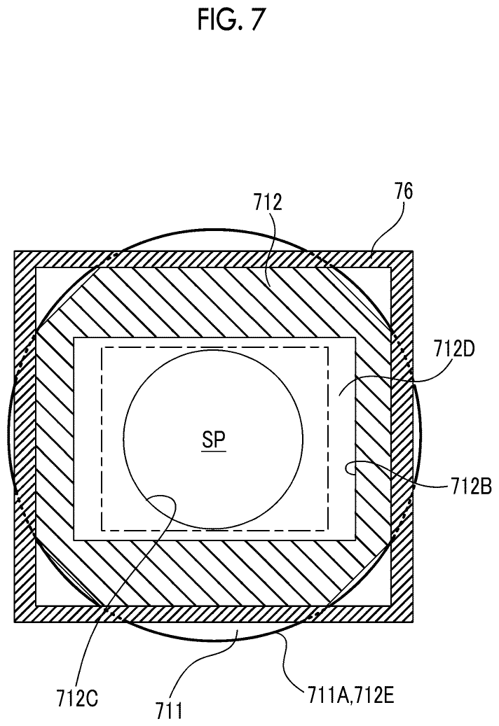

FIG. 3 shows an outer circumscribed circle 712E of the outer peripheral surface 712A of the rectangular tubular portion 712. As shown in FIG. 3, a diameter of the circumscribed circle 712E is larger than the outer diameter Da (a diameter of the outer peripheral surface 711A in FIG. 3, see FIG. 2) of the circular tubular portion 711. However, as shown in FIG. 7, the diameter of the circumscribed circle 712E may be the same as the outer diameter of the circular tubular portion 711. FIG. 7 is a schematic view showing a modification example of a B-B cross section of FIG. 2.

The second circuit substrate 74 is provided on the proximal end side with respect to the first circuit substrate 73 and has a third substrate surface 741 that intersects with (is perpendicular to, in the example of FIG. 2) the first substrate surface 731 and a fourth substrate surface 742 (a substrate surface perpendicular to the first substrate surface 731 in the example of FIG. 2) on a side opposite to the third substrate surface 741.

The second circuit substrate 74 is connected to the second substrate surface 732 of the first circuit substrate 73 on each of the third substrate surface 741 and the fourth substrate surface 742. The second circuit substrate 74 is a rigid substrate having a bending rigidity larger than that of the imaging element 72, and, for example, has a plate shape in which a plurality of wiring lines are laminated via an insulating layer from the third substrate surface 741 toward the fourth substrate surface 742. As the second circuit substrate 74, for example, a ceramic substrate, a glass substrate, a silicon substrate, or the like is used.

A plurality of terminals 73t are provided on the second substrate surface 732 of the first circuit substrate 73. In addition, a terminal 74t is provided on each of the third substrate surface 741 and the fourth substrate surface 742 of the second circuit substrate 74. The terminal 73t and the terminal 74t are electrically and mechanically connected to each other by solder Hb.

A terminal (not shown) electrically connected to the terminal 74t is provided on the third substrate surface 741 of the second circuit substrate 74, and a wiring line is connected to the terminal. A terminal (not shown) electrically connected to the terminal 74t is provided on the fourth substrate surface 742 of the second circuit substrate 74, and a wiring line is connected to the terminal. The wiring lines connected to the terminals of the third substrate surface 741 and the fourth substrate surface 742 are bundled to form a cable 75 and extend to the universal cord 4.

It should be noted that the second circuit substrate 74 may be connected to the second substrate surface 732 of the first circuit substrate 73 only on any one of the third substrate surface 741 or the fourth substrate surface 742. In addition, the second circuit substrate 74 may be configured to be connected to the cable 75 only on any one of the third substrate surface 741 or the fourth substrate surface 742.

At least one of the length 73X or the length 73Y of the first circuit substrate 73 may be the same as a length 74L of the second circuit substrate 74 in the axial direction (length in a direction along the third substrate surface 741 and the fourth substrate surface 742, see FIG. 2) but is preferably different therefrom. For example, the length 74L is larger than at least one of the length 73X or the length 73Y, so that the cable 75 is easily connected to the second circuit substrate 74. In addition, in a case where the length 74L is smaller than at least one of the length 73X or the length 73Y, a posture of the second circuit substrate 74 with respect to the first circuit substrate 73 can be easily stabilized.

A thickness 73D (see FIG. 2) of the first circuit substrate 73 and a thickness 74D (see FIG. 2) of the second circuit substrate 74 may be the same as each other, but are preferably different from each other. For example, the thickness 74D of the second circuit substrate 74 is smaller than the thickness 73D of the first circuit substrate 73, so that a space around the second circuit substrate 74 can be effectively utilized. In addition, in a case where the thickness 74D of the second circuit substrate 74 is larger than the thickness 73D of the first circuit substrate 73, rigidity of a connection body between the first circuit substrate 73 and the second circuit substrate 74 can be increased, and durability can be improved.

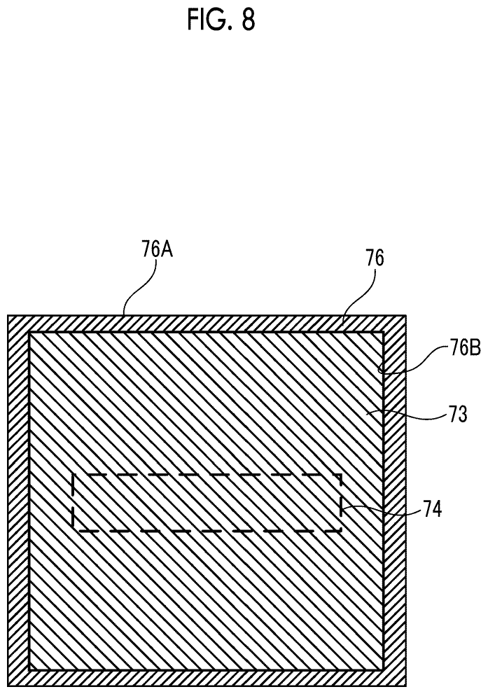

FIG. 8 is a schematic cross-sectional view taken along line C-C of FIG. 2. As shown in FIG. 8, the second circuit substrate 74 is disposed substantially at a center of the first circuit substrate 73 in a direction along the short side of the imaging element 72. The present disclosure is not limited to such an arrangement, and, for example, the second circuit substrate 74 may be disposed at one end portion of the first circuit substrate 73 in a direction along the short side of the imaging element 72.

As shown in FIGS. 4 and 8, the covering member 76 forms, for example, a rectangular tubular shape of which a cross-sectional shape perpendicular to the axial direction is a square frame.

The covering member 76 is made of, for example, metal. As shown in FIG. 2, the covering member 76 surrounds the rectangular tubular portion 712 of the support member 71, the imaging element 72, the first circuit substrate 73, the second circuit substrate 74, and the cable 75, and covers them.

As shown in FIG. 4, an inner peripheral surface 76B of the covering member 76 is directly or indirectly in contact with the outer peripheral surface 712A of the rectangular tubular portion 712. For example, the rectangular tubular portion 712 may be fitted into an inside of the covering member 76 so that the outer peripheral surface 712A is directly in contact with the inner peripheral surface 76B. Alternatively, the outer peripheral surface 712A of the rectangular tubular portion 712 may be adhered to the inner peripheral surface 76B with an adhesive so that the outer peripheral surface 712A is indirectly in contact with the inner peripheral surface 76B. In this way, the outer peripheral surface 712A of the rectangular tubular portion 712 is directly or indirectly in contact with the inner peripheral surface 76B of the covering member 76 so that the rectangular tubular portion 712 can be reinforced.

As shown in FIG. 8, the inner peripheral surface 76B of the covering member 76 is directly or indirectly in contact with each of four side surfaces of the first circuit substrate 73. For example, the first circuit substrate 73 may be fitted into the inside of the covering member 76 so that a side surface of the first circuit substrate 73 is directly in contact with the inner peripheral surface 76B. In addition, the side surface of the first circuit substrate 73 may be adhered to the inner peripheral surface 76B with an adhesive so that the side surface of the first circuit substrate 73 is indirectly in contact with the inner peripheral surface 76B. In this way, the side surface of the first circuit substrate 73 is directly or indirectly in contact with the inner peripheral surface 76B of the covering member 76 so that the first circuit substrate 73 can be reinforced.

In a case where the length 73X shown in FIG. 6 is the same as the length 72X, the inner peripheral surface 76B of the covering member 76 is directly or indirectly in contact with each of two side surfaces (side surfaces along the long side of the imaging element 72) of the first circuit substrate 73. In a case where the length 73Y shown in FIG. 6 is the same as the length 72Y, the inner peripheral surface 76B of the covering member 76 is directly or indirectly in contact with each of two side surfaces (side surfaces along the short side of the imaging element 72) of the first circuit substrate 73. Even in such a case, the first circuit substrate 73 can be reinforced.

FIG. 4 shows an outer diameter Dc of the covering member 76. The outer diameter Dc is larger than the maximum diameter Db (see FIG. 2) of the imaging optical system 70. In addition, the circumscribed circle 712E (see FIG. 3) of the rectangular tubular portion 712 is larger than the outer diameter Da of the circular tubular portion 711. As a result, as shown in FIG. 2, a clearance CL is provided between the covering member 76 or the rectangular tubular portion 712 and the distal end member 78.

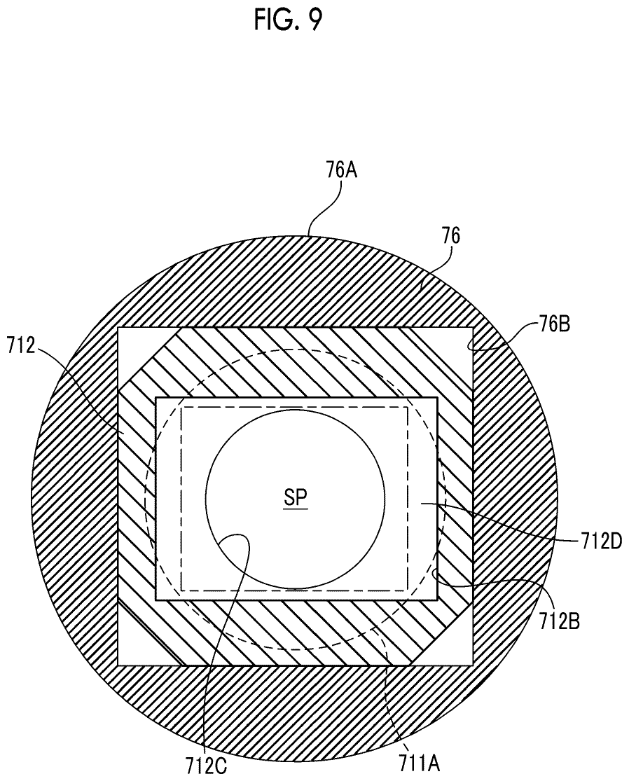

FIG. 9 is a schematic view showing a modification example of the covering member 76 and is a view corresponding to FIG. 4. As shown in FIG. 9, a cross-sectional shape of the outer peripheral surface 76A of the covering member 76 may be circular instead of rectangular.

The distal end portion 7 of the endoscope 1 configured as described above is manufactured, for example, as follows. First, an assembly in which the support member 71, the imaging element 72, the first circuit substrate 73, the second circuit substrate 74, the cable 75, and the covering member 76 are assembled is manufactured. Next, the imaging optical system 70 is inserted into the circular tubular portion 711 of the support member 71 of the assembly, and the imaging optical system 70 is fixed to the circular tubular portion 711 by using a captured image of the imaging element 72 to adjust an optical position between the imaging element 72 and the imaging optical system 70. Next, the assembly in which the imaging optical system 70 is fixed is inserted into the hole portion 781 of the distal end member 78, and the circular tubular portion 711 and the distal end member 78 are fixed in a state in which a distal end surface of the imaging optical system 70 and a distal end surface of the distal end member 78 are matched.

The optical position between the imaging element 72 and the imaging optical system 70 may vary due to assembly errors of the assembly, individual differences between the imaging element 72 and the imaging optical system 70, and the like. For example, in FIG. 2, assuming a case where the imaging optical system 70 is located closer to the imaging element 72, it is necessary to fix the circular tubular portion 711 and the distal end member 78 in a state in which the covering member 76 and an end surface of the rectangular tubular portion 712 on the distal end side are brought closer to the distal end member 78. Since the clearance CL is provided in the distal end portion 7, the distal end surface of the imaging optical system 70 and the distal end surface of the distal end member 78 can be matched even in a case where a variation in the optical position between the imaging element 72 and the imaging optical system 70 occurs.

In addition, in the endoscope 1, the outer peripheral surface 712A of the rectangular tubular portion 712 is in contact with the inner peripheral surface 76B of the covering member 76. Therefore, a unit including the support member 71, the imaging element 72, the first circuit substrate 73, the second circuit substrate 74, and the cable 75 can be protected, and durability can be improved. An inside of the support member 71 is filled with an adhesive such as an insulating epoxy-based resin to ensure water-tightness of the imaging element 72 and the like.

In addition, in the endoscope 1, the side surface of the first circuit substrate 73 is in contact with the inner peripheral surface 76B of the covering member 76. In addition, the imaging element 72 is not in contact with the covering member 76. Therefore, the imaging element 72 between the support member 71 and the first circuit substrate 73 can be more firmly protected. In particular, in a case where the total area of the plurality of terminals 73T is 30% or less of the area of the end surface 72A of the imaging element 72, mechanical connection strength between the imaging element 72 and the first circuit substrate 73 is lowered, so that a protective effect due to the contact between the rectangular tubular portion 712 and the first circuit substrate 73 and the covering member 76 can be enhanced. In addition, in a case where the area of the peripheral region 72a of the imaging element 72 is 50% or less of the area of the end surface 72A, mechanical connection strength between the imaging element 72 and the rectangular tubular portion 712 is lowered, so that a protective effect due to the contact between the rectangular tubular portion 712 and the covering member 76 can be enhanced.

In addition, in the endoscope 1, the covering member 76 covers the entire second circuit substrate 74. Therefore, even in a case where strength of the second circuit substrate 74 is reduced, such as a case where the length 74L of the second circuit substrate 74 is large and a case where the thickness 74D of the second circuit substrate 74 is small, the second circuit substrate 74 can be reinforced by the covering member 76.

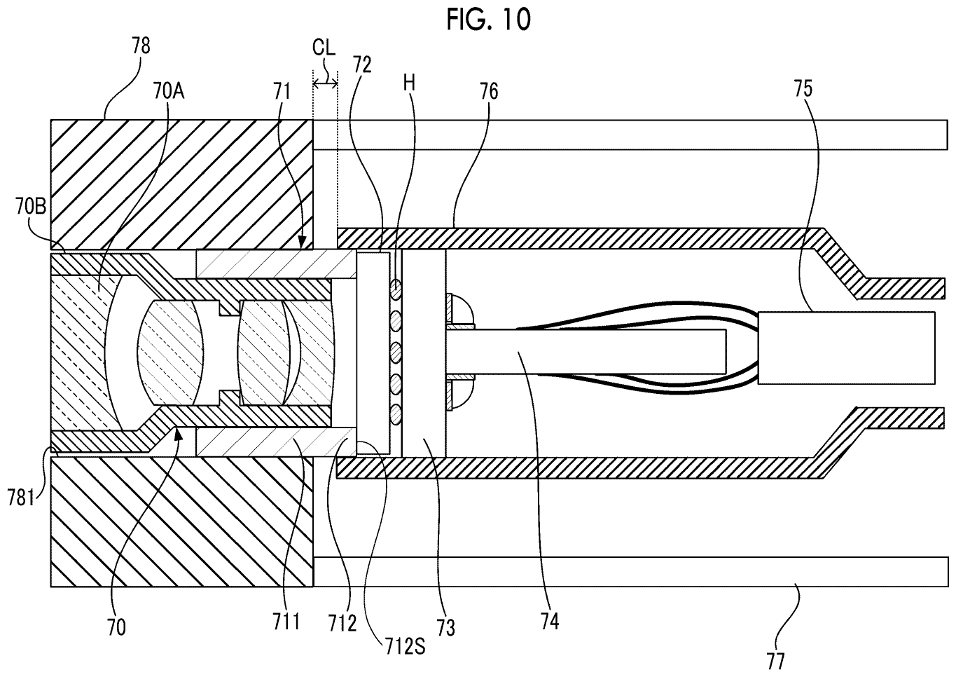

FIG. 10 is a schematic cross-sectional view showing a first modification example of the distal end portion 7. In FIG. 10, a difference from FIG. 2 is that the outer diameter Da of the circular tubular portion 711 and the diameter of the circumscribed circle 712E of the rectangular tubular portion 712 are the same, and the imaging element 72 is supported by an end surface 712S of the rectangular tubular portion 712 on the proximal end side.

In the modification example shown in FIG. 10, since a thickness of the rectangular tubular portion 712 is thin, a configuration is adopted in which the imaging element 72 is supported by the end surface 712S of the rectangular tubular portion 712 on the proximal end side. For example, the imaging element 72 is adhered to the end surface 712S of the rectangular tubular portion 712 on the proximal end side with an adhesive. In the modification example shown in FIG. 10, a clearance CL is provided between the covering member 76 and the distal end member 78. In the modification example shown in FIG. 10, the outer diameter of the covering member 76 can be made smaller than that of the configuration of FIG. 2, so that the insertion part 2 can be made smaller in diameter.

In the modification example shown in FIG. 10, it is preferable that a side surface of the imaging element 72 is not in contact with the covering member 76. As a result, it is possible to prevent a force from being directly applied to the imaging element 72 from the covering member 76.

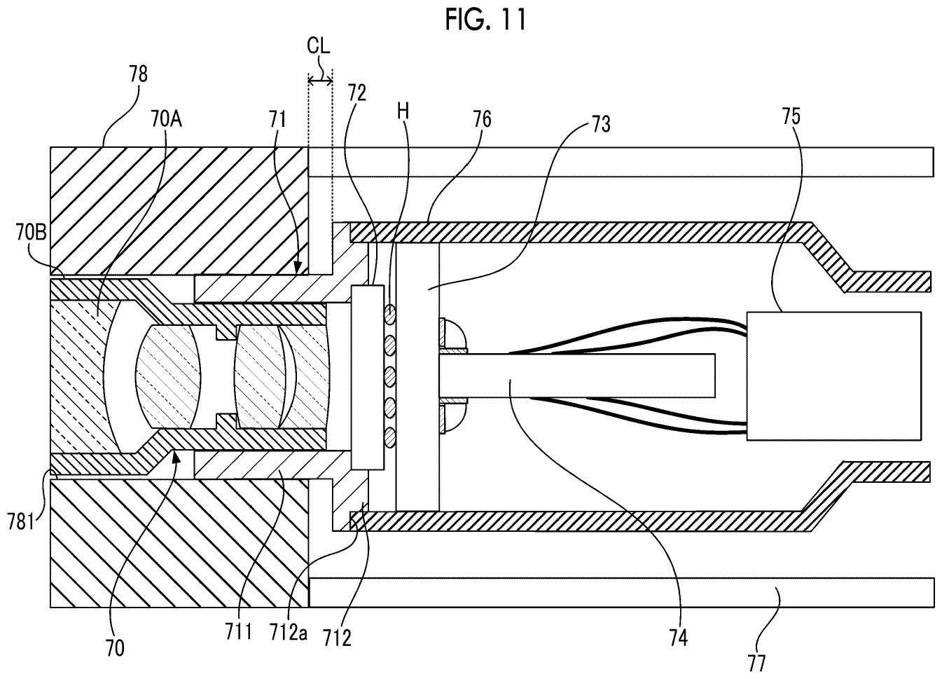

FIG. 11 is a schematic cross-sectional view showing a second modification example of the distal end portion 7. In FIG. 11, a difference from FIG. 2 is that a recessed portion 712a is added to an end portion of the rectangular tubular portion 712 on the proximal end side and an outer peripheral side, and a distal end portion of the covering member 76 is directly or indirectly in contact with the recessed portion 712a. That is, in the modification example shown in FIG. 11, the covering member 76 covers a part of the rectangular tubular portion 712, there is no clearance between the covering member 76 and the distal end member 78, and a clearance CL is provided between the rectangular tubular portion 712 and the distal end member 78. According to the modification example shown in FIG. 11, the outer diameter of the covering member 76 can be made smaller than that of the configuration shown in FIG. 2, so that the distal end portion 7 can be made smaller in diameter.

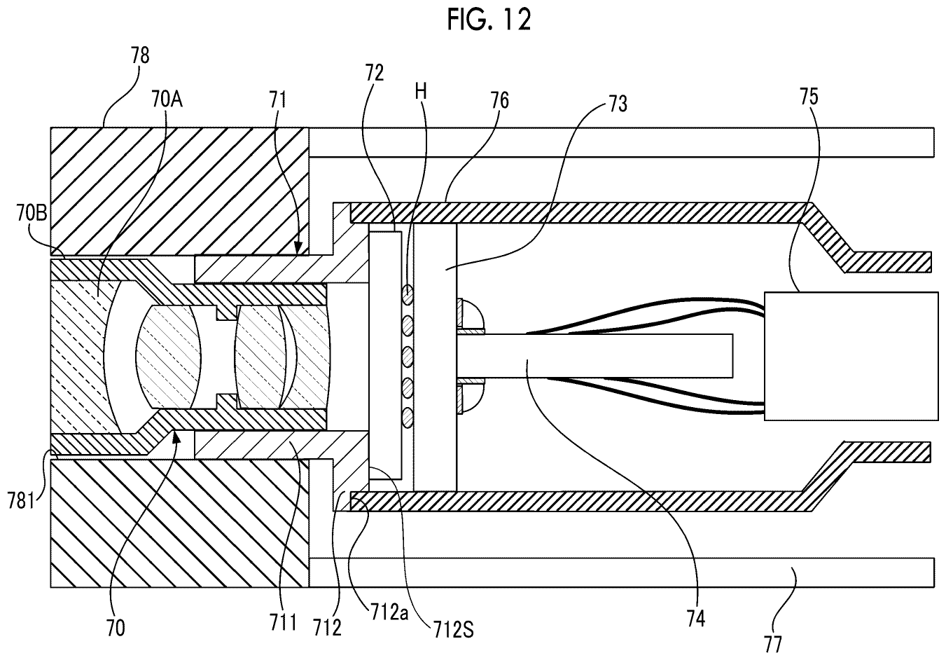

FIG. 12 is a schematic cross-sectional view showing a third modification example of the distal end portion 7. In FIG. 12, a difference from FIG. 2 is that the recessed portion 712a is added to the end portion of the rectangular tubular portion 712 on the proximal end side and the outer peripheral side, the distal end portion of the covering member 76 is directly or indirectly in contact with the recessed portion 712a, and the imaging element 72 is supported by the end surface 712S of the rectangular tubular portion 712 on the proximal end side. In the modification example shown in FIG. 12, there is no clearance between the covering member 76 and the distal end member 78, and a clearance CL is provided between the rectangular tubular portion 712 and the distal end member 78. According to the modification example shown in FIG. 12, the distal end portion 7 can be made smaller in diameter than that of the configuration shown in FIG. 2.

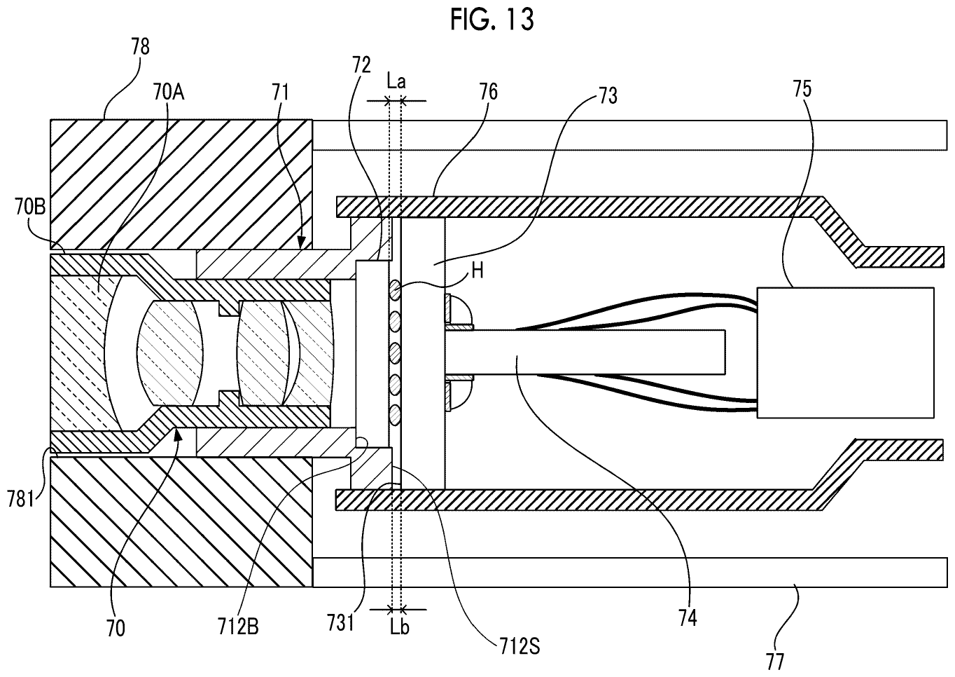

FIG. 13 is a schematic cross-sectional view showing a fourth modification example of the distal end portion 7. In FIG. 13, a difference from FIG. 2 is that the recessed portion 712B formed in the rectangular tubular portion 712 is formed deeper, and the imaging element 72, the first circuit substrate 73, and the second circuit substrate 74 are moved to the distal end side. In the modification example shown in FIG. 13, a distance Lb between the end surface 712S of the support member 71 on the proximal end side and the first substrate surface 731 of the first circuit substrate 73 is smaller than a distance La between the imaging element 72 and the first substrate surface 731 (in other words, a thickness of the solder ball H). With this configuration, for example, in a case where the end surface 712S of the rectangular tubular portion 712 and the first substrate surface 731 are adhered to each other with an adhesive, peeling of the adhesive can be suppressed.

In the configuration examples of the distal end portion 7 described so far, the second circuit substrate 74 is not essential and may be deleted. In this case, a configuration is adopted in which the terminals 73t of the first circuit substrate 73 and the cable 75 are connected to each other.

As described above, at least the following matters are described in the present specification. Hereinafter, constituent elements corresponding to the above-described embodiment are shown in parentheses, but the present disclosure is not limited thereto.

(1)

An endoscope comprising: an insertion part (insertion part 2) that is to be inserted into a subject, in which the insertion part includes an imaging optical system (imaging optical system 70), a tubular support member (support member 71) that supports the imaging optical system within the tubular support member, an imaging element (imaging element 72) that is provided on a proximal end side of the insertion part with respect to the imaging optical system and that is supported by the support member, a first circuit substrate (first circuit substrate 73) that has a first substrate surface (first substrate surface 731) facing a surface (end surface 72B) of the imaging element on the proximal end side of the insertion part and a second substrate surface (second substrate surface 732) on a side opposite to the first substrate surface and that is connected to the imaging element at the first substrate surface, a second circuit substrate (second circuit substrate 74) that is provided on the proximal end side of the insertion part with respect to the first circuit substrate, that has a third substrate surface (third substrate surface 741) intersecting with the first substrate surface and a fourth substrate surface (fourth substrate surface 742) on a side opposite to the third substrate surface, and that is connected to the second substrate surface at at least one of the third substrate surface or the fourth substrate surface, a cable (cable 75) that is connected to at least one of the third substrate surface or the fourth substrate surface of the second circuit substrate, a covering member (covering member 76) that covers a part of the support member, the imaging element, the first circuit substrate, the second circuit substrate, and the cable, and a distal end member (distal end member 78) having a hole portion (hole portion 781) into which the support member and the imaging optical system are inserted, the support member includes a circular tubular portion (circular tubular portion 711) having a circular tubular shape that supports the imaging optical system and a rectangular tubular portion (rectangular tubular portion 712) having a rectangular tubular shape that supports the imaging element, the circular tubular portion is supported by the hole portion of the distal end member, a clearance (clearance CL) is provided between the distal end member and the covering member or the rectangular tubular portion of the support member, the covering member covers at least a part of the rectangular tubular portion, and a diameter of a circumscribed circle (circumscribed circle 712E) of the rectangular tubular portion is equal to or larger than an outer diameter (outer diameter Da) of the circular tubular portion.

(2)

The endoscope according to (1), in which an outer diameter (maximum diameter Db) of the imaging optical system is smaller than the outer diameter (outer diameter Da) of the circular tubular portion.

(3)

The endoscope according to (1) or (2), in which the imaging element is supported by an end surface (end surface 712S) of the support member on the proximal end side of the insertion part, and a side surface of the first circuit substrate is directly or indirectly in contact with the covering member.

(4)

The endoscope according to (1) or (2), in which in a case of being viewed in an axial direction of the insertion part, a size (length 73X and length 73Y) of the first circuit substrate is larger than a size (outer diameter Df) of a portion of the imaging optical system which is supported by the support member.

(5)

The endoscope according to (4), in which a side surface of the first circuit substrate is directly or indirectly in contact with the covering member.

(6)

The endoscope according to (1) or (2), in which in a case of being viewed in an axial direction of the insertion part, a size of the first circuit substrate is larger than a size of the imaging element.

(7)

The endoscope according to (6), in which a side surface of the first circuit substrate is directly or indirectly in contact with the covering member.

(8)

The endoscope according to (1) or (2), in which a distance (distance Lb) between an end surface (end surface 712S) of the support member on the proximal end side of the insertion part and the first substrate surface of the first circuit substrate is smaller than a distance (distance La) between the imaging element and the first substrate surface.

(9)

The endoscope according to (1) or (2), in which in a case of being viewed in an axial direction of the insertion part, an outer diameter (outer diameter Dc) of the covering member is larger than an outer diameter (maximum diameter Db) of the imaging optical system.

(10)

The endoscope according to (1) or (2), in which a thickness (thickness 73D) of the first circuit substrate and a thickness (thickness 74D) of the second circuit substrate are different from each other.

(11)

The endoscope according to (10), in which the thickness of the second circuit substrate is smaller than the thickness of the first circuit substrate.

(12)

The endoscope according to (1) or (2), in which a first length (at least one of length 73X or length 74X) of the first circuit substrate along the first substrate surface and the second substrate surface and a second length (length 74L) of the second circuit substrate along the third substrate surface and the fourth substrate surface are different from each other.

(13)

The endoscope according to (12), in which the second length is larger than the first length.

(14)

The endoscope according to (1) or (2), in which a plurality of terminals (terminals 73T) electrically connected to the imaging element are provided on the first substrate surface, and in a case of being viewed in an axial direction of the insertion part, a total area of the plurality of terminals is 30% or less of an area of an end surface (end surface 72A) of the imaging element on a distal end side of the insertion part.

(15)

The endoscope according to (1) or (2), in which the imaging element is supported by the support member by adhesion, and an area of a region (peripheral region 72a) of the imaging element which is adhered to the support member is 50% or less of an area of an end surface (end surface 72A) of the imaging element on a distal end side of the insertion part.

(16)

An endoscope comprising: an insertion part (insertion part 2) that is to be inserted into a subject, in which the insertion part includes an imaging optical system (imaging optical system 70), a tubular support member (support member 71) that supports the imaging optical system within the tubular support member, an imaging element (imaging element 72) that is provided on a proximal end side of the insertion part with respect to the imaging optical system and that is supported by the support member, a first circuit substrate (first circuit substrate 73) that has a first substrate surface (first substrate surface 731) facing a surface (end surface 72B) of the imaging element on the proximal end side of the insertion part and a second substrate surface (second substrate surface 732) on a side opposite to the first substrate surface and that is connected to the imaging element at the first substrate surface, a cable (cable 75) that is provided on the proximal end side of the insertion part with respect to the first circuit substrate and that is electrically connected to the first circuit substrate, a covering member (covering member 76) that covers a part of the support member, the imaging element, the first circuit substrate, and the cable, and a distal end member (distal end member 78) having a hole portion (hole portion 781) into which the support member and the imaging optical system are inserted, the support member includes a circular tubular portion (circular tubular portion 711) having a circular tubular shape that supports the imaging optical system and a rectangular tubular portion (rectangular tubular portion 712) having a rectangular tubular shape that supports the imaging element, the circular tubular portion is supported by the hole portion of the distal end member, a clearance (clearance CL) is provided between the distal end member and the covering member or the rectangular tubular portion of the support member, a plurality of terminals (terminals 73T) electrically connected to the imaging element are provided on the first substrate surface, and in a case of being viewed in an axial direction of the insertion part, a total area of the plurality of terminals is 30% or less of an area of an end surface (end surface 72A) of the imaging element on a distal end side of the insertion part.

(17)

The endoscope according to (16), in which the imaging element is supported by the support member by adhesion, and an area of a region (peripheral region 72a) of the imaging element which is adhered to the support member is 50% or less of the area of the end surface (end surface 72A) of the imaging element on the distal end side of the insertion part.

(18)

The endoscope according to (16) or (17), further comprising: a second circuit substrate (second circuit substrate 74) that is provided on the proximal end side of the insertion part with respect to the first circuit substrate, that has a third substrate surface (third substrate surface 741) intersecting with the first substrate surface and a fourth substrate surface (fourth substrate surface 742) on a side opposite to the third substrate surface, and that is connected to the second substrate surface at at least one of the third substrate surface or the fourth substrate surface, in which the cable is connected to at least one of the third substrate surface or the fourth substrate surface of the second circuit substrate, and the covering member covers a part of the support member, the imaging element, the first circuit substrate, the second circuit substrate, and the cable.

(19)

The endoscope according to (18), in which the covering member covers at least a part of the rectangular tubular portion.

(20)

The endoscope according to (19), in which a diameter of a circumscribed circle (circumscribed circle 712E) of the rectangular tubular portion is equal to or larger than an outer diameter (outer diameter Da) of the circular tubular portion.

(21)

The endoscope according to (20), in which an outer diameter (maximum diameter Db) of the imaging optical system is smaller than the outer diameter (outer diameter Da) of the circular tubular portion.

(22)

The endoscope according to (18), in which the imaging element is supported by an end surface (end surface 712S) of the support member on the proximal end side of the insertion part, and a side surface of the first circuit substrate is directly or indirectly in contact with the covering member.

(23)

The endoscope according to (18), in which in a case of being viewed in the axial direction of the insertion part, a size (length 73X and length 73Y) of the first circuit substrate is larger than a size (outer diameter Df) of a portion of the imaging optical system which is supported by the support member.

(24)

The endoscope according to (23), in which a side surface of the first circuit substrate is directly or indirectly in contact with the covering member.

(25)

The endoscope according to (18), in which in a case of being viewed in the axial direction of the insertion part, a size of the first circuit substrate is larger than a size of the imaging element.

(26)

The endoscope according to (25), in which a side surface of the first circuit substrate is directly or indirectly in contact with the covering member.

### EXPLANATION OF REFERENCES

1: endoscope
2: insertion part
3: operating part
4: universal cord
5: soft portion
6: bendable portion
7: distal end portion
8, 9: angle knob
10: air/water supply button
12: treatment tool inlet port
70: imaging optical system
70B: lens frame
71: support member
72: imaging element
72A, 72B, 712S: end surface
72a: peripheral region
73: first circuit substrate
73T, 73t, 74t: terminal
74: second circuit substrate
75: cable
76: covering member
76A, 711A, 712A: outer peripheral surface
76B, 711B: inner peripheral surface
77: exterior member
78: distal end member
711: circular tubular portion
712: rectangular tubular portion
712B, 712a: recessed portion
712C: opening
712D: adhesive region
712E: circumscribed circle
731: first substrate surface
732: second substrate surface
741: third substrate surface
742: fourth substrate surface
781: hole portion

What is claimed is:

1. An endoscope comprising:
an insertion part that is to be inserted into a subject,
wherein the insertion part includes an imaging optical system,
a tubular support member that supports the imaging optical system within the tubular support member,
an imaging element that is provided on a proximal end side of the insertion part with respect to the imaging optical system and that is supported by the support member, a first circuit substrate that has a first substrate surface facing a surface of the imaging element on the proximal end side of the insertion part and a second substrate surface on a side opposite to the first substrate surface and that is connected to the imaging element at the first substrate surface,
a second circuit substrate that is provided on the proximal end side of the insertion part with respect to the first circuit substrate, that has a third substrate surface intersecting with the first substrate surface and a fourth substrate surface on a side opposite to the third substrate surface, and that is connected to the second substrate surface at at least one of the third substrate surface or the fourth substrate surface,
a cable that is connected to at least one of the third substrate surface or the fourth substrate surface of the second circuit substrate,
a covering member that covers a part of the support member, the imaging element, the first circuit substrate, the second circuit substrate, and the cable, and
a distal end member having a hole portion into which the support member and the imaging optical system are inserted,
the support member includes a circular tubular portion having a circular tubular shape that supports the imaging optical system and a rectangular tubular portion having a rectangular tubular shape that supports the imaging element,
the circular tubular portion is supported by the hole portion of the distal end member, a clearance is provided between the distal end member and the covering member or the rectangular tubular portion of the support member,
the covering member covers at least a part of the rectangular tubular portion, and
a diameter of a circumscribed circle of the rectangular tubular portion is equal to or larger than an outer diameter of the circular tubular portion.

2. The endoscope according to claim 1,
wherein an outer diameter of the imaging optical system is smaller than the outer diameter of the circular tubular portion.

3. The endoscope according to claim 1,
wherein the imaging element is supported by an end surface of the support member on the proximal end side of the insertion part, and
a side surface of the first circuit substrate is directly or indirectly in contact with the covering member.

4. The endoscope according to claim 1,
wherein in a case of being viewed in an axial direction of the insertion part, a size of the first circuit substrate is larger than a size of a portion of the imaging optical system which is supported by the support member.

5. The endoscope according to claim 4,
wherein a side surface of the first circuit substrate is directly or indirectly in contact with the covering member.

6. The endoscope according to claim 1,
wherein in a case of being viewed in an axial direction of the insertion part, a size of the first circuit substrate is larger than a size of the imaging element.

7. The endoscope according to claim 6,
wherein a side surface of the first circuit substrate is directly or indirectly in contact with the covering member.

8. The endoscope according to claim 1,
wherein a distance between an end surface of the support member on the proximal end side of the insertion part and the first substrate surface of the first circuit substrate is smaller than a distance between the imaging element and the first substrate surface.

9. The endoscope according to claim 1,
wherein in a case of being viewed in an axial direction of the insertion part, an outer diameter of the covering member is larger than an outer diameter of the imaging optical system.

10. The endoscope according to claim 1,
wherein a thickness of the first circuit substrate and a thickness of the second circuit substrate are different from each other.

11. The endoscope according to claim 10,
wherein the thickness of the second circuit substrate is smaller than the thickness of the first circuit substrate.

12. The endoscope according to claim 1,
wherein a first length of the first circuit substrate along the first substrate surface and the second substrate surface and a second length of the second circuit substrate along the third substrate surface and the fourth substrate surface are different from each other.

13. The endoscope according to claim 12,
wherein the second length is larger than the first length.

14. The endoscope according to claim 1,
wherein a plurality of terminals electrically connected to the imaging element are provided on the first substrate surface, and
in a case of being viewed in an axial direction of the insertion part, a total area of the plurality of terminals is 30% or less of an area of an end surface of the imaging element on a distal end side of the insertion part.

15. The endoscope according to claim 1,
wherein the imaging element is supported by the support member by adhesion, and
an area of a region of the imaging element which is adhered to the support member is 50% or less of an area of an end surface of the imaging element on a distal end side of the insertion part.
